(19) United States
(12) Patent Application Publication
(10) Pub. No.: US 2016/0058500 A1
(43) Pub. Date: Mar. 3, 2016

(54) REMOVING TISSUE WITH ELECTROSURGICAL LOOP AND SUCTION

(71) Applicant: COVIDIEN LP, MANSFIELD, MA (US)

(72) Inventors: ROBERT M. SHARP, BOULDER, CO (US); REBECCA J. COULSON, LYONS, CO (US)

(21) Appl. No.: 14/801,179

(22) Filed: Jul. 16, 2015

Related U.S. Application Data
(60) Provisional application No. 62/042,528, filed on Aug. 27, 2014.

(51) Int. Cl. A61B 18/14

(52) U.S. Cl. CPC A61B 18/1485; A61B 2018/00601

(57) ABSTRACT

An electrosurgical instrument for removing tissue includes a shaft and a cutting member. The shaft extends to a distal end and defines a lumen configured to communicate with a vacuum source. The cutting member is supported at the distal end of the shaft. The cutting member is adapted to couple to an electrosurgical energy source and is configured to form an annular loop. The cutting member is selectively transitionable from a first configuration, wherein the annular loop includes a first diameter to one or more additional configurations, wherein the annular loop includes a second diameter smaller than the first diameter. The annular loop is configured to sever tissue within the annular loop.

### CROSS REFERENCE TO RELATED APPLICATION

The present application claims the benefit of and priority to U.S. Provisional Application Ser. No. 62/042,528, filed on Aug. 27, 2014, the entire contents of which are incorporated herein by reference.

### TECHNICAL FIELD

The present disclosure relates to surgical devices, systems, and methods for performing tonsillectomies and adenoidectomies, and, more particularly, to surgical devices systems, and methods with loop and suction features utilized for tonsillectomies and adenoidectomies.

### BACKGROUND

The tonsils and adenoids are part of the lymphatic system and are generally located in the back of the throat. These parts of the lymphatic system are generally used for sampling bacteria and viruses entering the body and activating the immune system when warranted to produce antibodies to fight oncoming infections. More particularly, the tonsils and adenoids break down the bacteria or virus and send pieces of the bacteria or virus to the immune system to produce antibodies for fighting off infections.

Inflammation of the tonsils and adenoids (e.g., tonsillitis) impedes the ability of the tonsils and adenoids to destroy the bacteria resulting in a bacterial infection. In many instances, the bacteria remain even after treatment and serve as a reservoir for repeated infections (e.g., tonsillitis or ear infections).

A tonsillectomy and/or adenoidectomy may be indicated when infections persist and antibiotic treatments fail. Persistent infection typically leads to enlarged tonsil tissue which may need to be removed since in many cases the enlarged tissue causes airway obstruction leading to various sleep disorders such as snoring or, in some cases, sleep apnea. Some individuals are also born with larger tonsils that are more prone to cause obstruction. An adenoidectomy may also be required to remove adenoid tissue when ear pain persists, or when nose breathing or function of the eustachian tube (a.k.a. auditory or pharyngotympanic tube) is impaired. Many surgeons prefer to perform these two procedures at the same time.

The type of surgical device and/or method used for tonsillectomies and adenoidectomies usually depends on the type and amount of tissue to be removed and/or a surgeon's preference. Various methods for performing a tonsillectomy and/or adenoidectomy employ an array of surgical instrumentation and, in most cases, a variety of energy modalities to accomplish the underlying purpose of removing the infected tissue. Technologies include: cold dissection, monopolar and bipolar diathermy dissection, dissection using bipolar scissors, laser tonsillectomy, cryosurgery, ultrasonic removal, microdebrider, Coblation® and so-called thermal welding. A scalpel or other sharp instrument such as a curette or punch device may also be used to remove tissue but using these types of instruments typically results in heavy bleeding which needs to be stemmed with electrocautery.

Other electrosurgical devices may also be employed such as suction-tipped devices, blades, or needle tip devices, e.g., various Bovie devices. A Bovie device typically has a hollow center to suction blood, secretions, and smoke from the surgical field, and a rim of metal for cutting and coagulation. A separate aspirator is used when blade and needle tip Bovies are used. Although the use of Bovies reduces blood loss intraoperatively in comparison to various known cold techniques, its use may be associated with an increase in postoperative pain due to thermal spread from the heat created during use (e.g., above 3000° C.). Despite a large thermal injury profile, use of the Bovie surgical instrument remains very popular for tonsil removal.

Some commercial attempts have been made to limit or minimize thermal injury. These include: the Harmonic Scalpel® system (Ethicon Endo-Surgery, Cincinnati, Ohio) (ultrasonic energy), lasers (e.g., KTP, Nd: YAG, or CO2 lasers), and Coblation® devices (Arthrocare, Austin, Tex.) (bipolar radiofrequency ablation). However, the decrease in thermal injury provided by these devices remains questionable and is offset by a reduced control of bleeding and surrounding tissue trauma, longer operative times, introduction of fluids (e.g., saline) and/or less precise cutting. Some of the instruments also obscure the surgical field and are difficult to maneuver due to their large size.

Moreover many of these devices are not suited to treat or remove the underlying tissue or tissue within the tonsil bed which, in many instances, can lead to serious concerns. As mentioned above, the tonsil bed, if not properly treated or removed, acts as a reservoir for bacteria leading to repeated infections.

### SUMMARY

Accordingly, new devices for resecting tonsil and adenoid tissue would be useful. In particular, devices that precisely cut tonsil and adenoid tissue while effectively controlling bleeding and surrounding tissue trauma would be desirable. Devices that provide easier access to the tonsils and adenoids and manipulation of those tissues would also be desirable.

In one aspect, the present disclosure relates to an electrosurgical instrument for removing tissue including a shaft and a cutting member. The shaft extends to a distal end and defines a lumen configured to communicate with a vacuum source.

The cutting member is supported at the distal end of the shaft. The cutting member is adapted to couple to an electrosurgical energy source and configured to form an annular loop. The cutting member is selectively transitionable from a first configuration, wherein the annular loop includes a first diameter, to one or more additional configurations, wherein the annular loop includes a second diameter smaller than the first diameter. A portion of the cutting member extends through the shaft and is axially movable relative to the shaft to transition the annular loop from the first diameter to the second diameter.

In some embodiments, a portion of the cutting member may include an insulative material. At least a portion of the shaft may be formed of an insulative material. In certain embodiments, the shaft may be at least partially formed from an electrically-conductive material that is insulated from at least the annular loop of the cutting member. The electrically-conductive material of the shaft may be configured to provide a return path for electrosurgical energy communicated to the annular loop of the cutting member.

The annular loop is configured to sever tissue within the annular loop. The annular loop may form a monopolar electrode. In some embodiments, the annular loop may be configured to electrically communicate with a return pad in electrical communication with the electrosurgical energy source. The annular loop may define an annular cutting surface configured to at least partially circumferentially cut through tissue in response to the transitioning of the annular loop. The annular loop defines a central opening having a central axis therethrough. The central axis may be parallel to, or coaxial with, a longitudinally axis defined by the shaft.

According to one aspect, an electrosurgical system for removing tissue includes an electrosurgical energy source, a vacuum source, a shaft, and a cutting member.

In yet another aspect, a method for removing tissue is provided. The method involves encircling tissue with an annular loop disposed at a distal end of a shaft, the annular loop including an initial diameter; activating the annular loop with electrosurgical energy to circumferentially cut the tissue encircled therein; transitioning the diameter of the annular loop to facilitate severance of the tissue; and aspirating the tissue via the shaft. The method may involve removing the tissue from a throat with monopolar energy, bipolar energy, or combinations of bipolar and monopolar energy. The tissue may include tonsil tissue, adenoid tissue, or combinations of tonsil and adenoid tissue.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1A is a perspective view of one embodiment of an electrosurgical instrument according to the principles of the present disclosure;

FIG. 1B is a schematic, perspective view of a motor of an embodiment of the electrosurgical instrument of FIG. 1A;

FIGS. 2A-2C are progressive views illustrating various conditions of a cutting member of the electrosurgical instrument of FIG. 1A;

FIGS. 3A-3D are progressive views illustrating the electrosurgical instrument of FIG. 1A removing throat tissue from a throat;

FIGS. 4A-4C are progressive views illustrating various conditions of a cutting member of another embodiment of the electrosurgical instrument of FIG. 1A; and

FIGS. 5A-5C are progressive views illustrating various conditions of a cutting member of yet another embodiment of the electrosurgical instrument of FIG. 1A.

### DETAILED DESCRIPTION

Particular embodiments of the present disclosure are described hereinbelow with reference to the accompanying drawings in which like reference numerals designate identical or corresponding elements in each of the several views. As used herein the term “distal” refers to that portion of the system, apparatus and/or device, or component thereof, that are farther from the user, while the term “proximal” refers to that portion of the system, apparatus and/or device, or component thereof, that are closer to the user. In the following description, well-known functions or constructions are not described in detail to avoid obscuring the present disclosure in unnecessary detail.

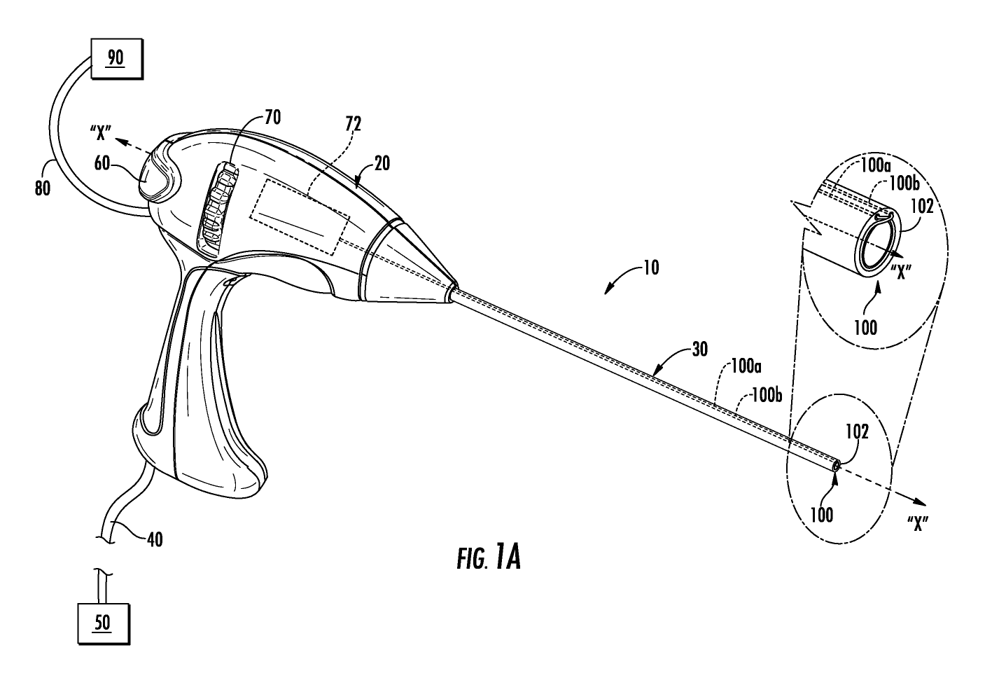

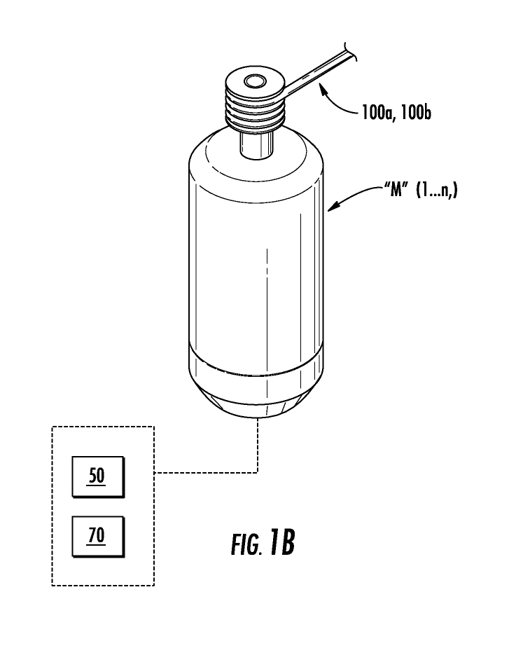

Turning now to FIG. 1A, an electrosurgical instrument 10, which may be monopolar and/or bipolar, is provided. Electrosurgical instrument 10 defines a longitudinal axis “X” and includes a handle assembly 20, a shaft 30 that extends distally from handle assembly 20, and a cutting member 100 that extends distally through shaft 30, from first and second proximal portions 100a, 100b, to an annular loop 102 supported on a distal end of shaft 30. In some embodiments, shaft 30 may be curved. Electrosurgical instrument 10 includes a cable 40 that connects electrosurgical instrument 10 to an energy source 50 (e.g., a generator 50 or other suitable power source). In some embodiments, electrosurgical instrument 10 may be configured as a battery-powered device. Cable 40 includes a wire (or wires) that extend therethrough and that connect to cutting member 100 to provide energy thereto. In one embodiment, cutting member 100 is a monopolar element/electrode.

An activation switch 60 is supported on handle assembly 20 for selectively supplying energy to cutting member 100 and a loop adjustment actuator 70 is supported on handle assembly 20 for adjusting a diameter of annular loop 102 as will be described in greater detail below. Loop adjustment actuator 70 can be operatively coupled to first and/or second proximal portions 100a, 100b of cutting member 100 via a drive assembly 72 supported in handle assembly 20 to adjust the diameter of annular loop 102 upon actuation of loop adjustment actuator 70.

Activation switch 60 and/or loop adjustment actuator 70 can include any suitable actuator (e.g., a trigger, button, slide, wheel, etc.) that couples to drive assembly 72. Drive assembly 72 can include any suitable mechanical and/or electrical component (e.g., gears, pulleys, circuitry, controllers, piezo technology, etc.) (not shown) that cooperate to manually and/or autonomously effectuate the functions of activation switch 60 and/or loop adjustment actuator 70 described herein.

For example, in one manually operated embodiment, first and/or second proximal portions 100a, 100b of cutting member 100 can be coupled to one or more gears and/or pulleys (not shown) of drive assembly 72 for winding-up and/or letting out a length of cutting member 100 to adjust the diameter of annular loop 102 upon actuation of loop adjustment actuator 70. In yet another example, as seen in FIG. 1B, drive assembly 72 can include one or more motors “M” that are operatively coupled to first and/or second proximal portions 100a, 100b of cutting member 100 for autonomously adjusting the diameter of annular loop 102 upon actuation of loop adjustment actuator 70. Specifically, each motor “M” may be configured to wind up or let out a length of cutting member 100 to adjust the diameter of annular loop 102. Although FIG. 1B shows a single cutting member that is wound up or let out by a single motor, cutting member 100 and/or one or more motors “M” can have any suitable configuration. In some instances, two or more cutting members 100 and/or two ends/portions of a single cutting member 100 may be wound up or let out by a single motor and/or multiple motors.

As seen in FIG. 1A, handle assembly 20 includes a vacuum conduit 80 disposed in fluid communication with a vacuum/suction source 90. Vacuum conduit 80 is disposed in fluid communication with shaft 30 to enable suction source 90 to apply suction/aspiration through shaft 30.

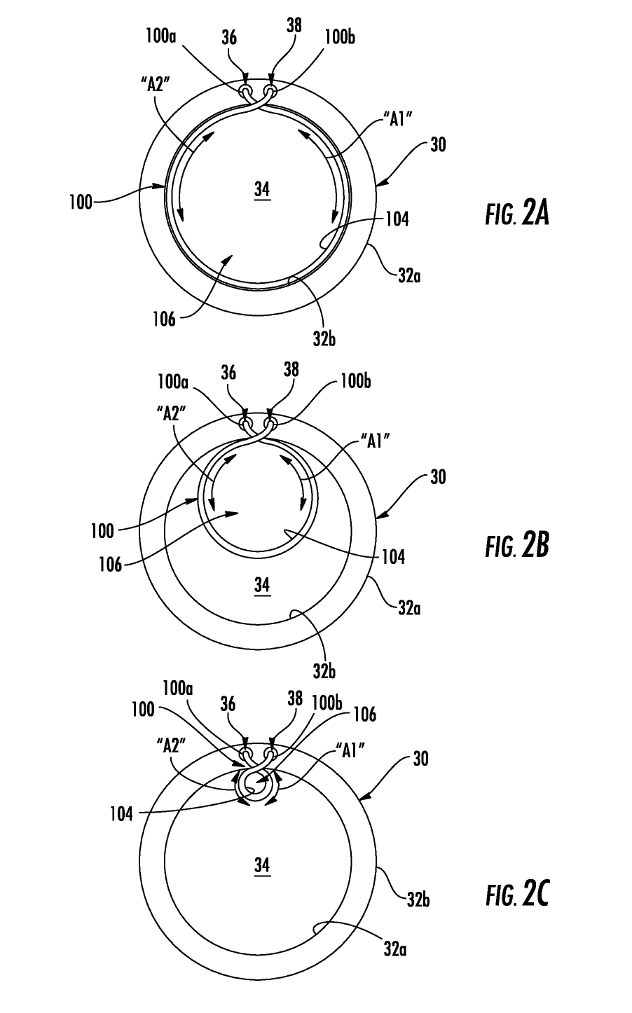

Referring to FIGS. 2A-2C, shaft 30 includes an outer surface 32a and an inner surface 32b. Inner surface 32b defines a suction lumen 34 that extends therethrough and that is in fluid communication with suction source 90 (FIG. 1A). Shaft 30 defines a first wire lumen 36 and a second wire lumen 38 that extend therethrough and are configured to receive first and second proximal portions 100a, 100b of cutting member 100, respectively. Shaft 30 and/or cutting member 100 can be wholly, or partially, formed of electrically conductive material. In embodiments, shaft 30 and/or cutting member 100 is wholly, or partially, formed of insulative/dielectric material. In some embodiments, insulative/dielectric material may be supported on electrically conductive material of shaft 30 and/or cutting member 100, for example, as a coating and/or layer. In certain embodiments, shaft 30 and/or cutting member 100, or portions thereof, may be configured to couple to the same and/or different electrical potentials, which may have the same and/or different polarities. In embodiments, shaft 30 and/or cutting member 100, or portions thereof, may be separated by a dielectric material.

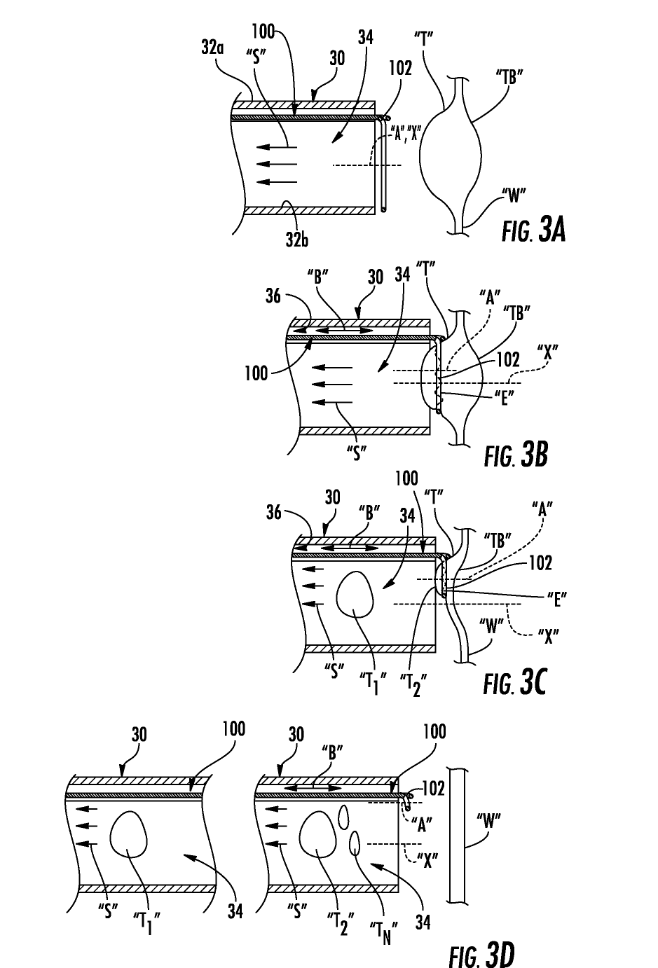

Annular loop 102 has a ring shape and defines an annular cutting surface 104 configured to at least partially circumferentially cut through tissue. Annular loop 102 defines a central opening 106 therethrough that is closable upon cinching annular loop 102. Central opening 106 defines a central axis “A” (see FIGS. 3A-3D) that can be positioned parallel to (FIGS. 3B-3D), or coaxial with (FIG. 3A), the longitudinal axis “X” of shaft 30. The cutting member 100 is selectively transitionable from a first configuration (see FIG. 2A) wherein annular loop 102 includes a diameter large enough to encircle tissue to at least one additional configuration (see FIGS. 2B and 2C) wherein annular loop 102 cinches tissue therein. Additional configurations may include smaller and/or larger diameter configurations. Annular loop 102 may be cinched completely closed so that the central opening is eliminated, or substantially eliminated, to facilitate severance of tissue.

In operation, as seen in FIGS. 3A-3D, shaft 30 is positioned in a first configuration adjacent a tissue wall “W” with annular loop 102 having a diameter large enough to encircle tissue “T” of the tissue wall “W.” In the illustrated embodiment, tissue “T” is throat tissue such as tonsil and/or adenoid tissue; however, the presently described devices, systems, and methods can be applied to any tissue.

As illustrated by arrow “B,” the diameter of annular loop 102 can be changed as desired for receiving and cinching tissue “T” of any suitable size therein. In particular, as illustrated by arrows “A1” and “A2” seen in FIGS. 2A-2C, the diameter of annular loop 102 can be transitioned to at least one additional configuration by proximally drawing and/or distally extending first and/or second proximal portions 102a, 102b of cutting member 100 axially through first and/or second wire lumens 36, 38 upon actuation of loop adjustment actuator 70 (FIG. 1A).

With vacuum source 90 activated, suction “S” provided through shaft 30 can draw tissue “T” into suction lumen 34 to accommodate tissue “T” within central opening 106 of annular loop 102. With annular loop 102 encircled about tissue “T” and cinched thereabout as desired, activation switch 60 (FIG. 1A) is actuated to communicate electrosurgical energy “E” to annular cutting surface 104 of annular loop 102. Electrosurgical energy “E” may include, for example, monopolar energy; however, any suitable energy arrangements and/or modalities can be used. With shaft 30 and/or a return pad (not shown) coupled to a first electrical potential of electrosurgical energy source 50 and annular loop 102 coupled to a second electrical potential of electrosurgical energy source 50, electrosurgical energy “E” can be conducted through annular cutting surface 104 of annular loop 102, through tissue “T,” and returned through shaft 30 and/or the return pad.

As electrosurgical energy “E” is conducted through annular cutting surface 104, annular cutting surface 104 circumferentially cuts through tissue “T” disposed within central opening 106 of annular loop 102. The diameter of annular loop 102 can be adjusted as necessary to enable severance of tissue “T” from a tissue bed “TB” of tissue wall “W.” At least portions of the tissue bed “TB” can also be removed as desired. Electrosurgical energy “E” may be applied simultaneously with, and/or independent from, diameter adjustments to annular loop 102. The tissue “T” may be severed unitarily or incrementally by tissue portions “T₁,” “T₂,” . . . “T_{N}.” Once severed from the tissue wall “W,” severed tissue may be aspirated through suction lumen 34 of shaft 30 for removal. This process, or portions thereof, can be repeated as necessary.

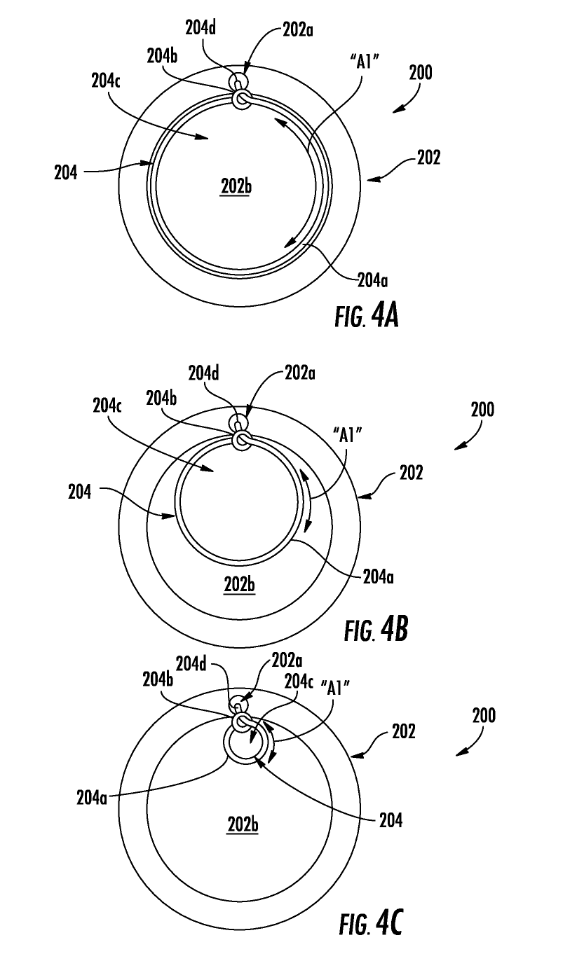

Advantageously, the various components of electrosurgical instrument 10 cooperate to provide easy access to and/or manipulation of tissue “T” and also enable a clinician to precisely cut tissue upon the application of electrosurgical energy “E” while effectively controlling bleeding and surrounding tissue trauma. FIGS. 4A-4C illustrate one embodiment an electrosurgical instrument 200 that is similar to electrosurgical instrument 10 and therefore is described herein only to the extent necessary to describe the differences in construction and operation thereof. Electrosurgical instrument 200 includes a shaft 202 and a cutting member 204. Shaft 202 defines a wire lumen 202a that receives a proximal portion of cutting member 204 and suction lumen 202b that is in fluid communication with fluid source 90 (FIG. 1A). Cutting member 204 extends distally to an annular loop 204a having a connector 204b and defining a central opening 204c. Connector 204b may have any suitable shape, such as a ring, through which portions of cutting member 204 are thread and/or otherwise coupled. Connector 204 may be formed of a dielectric material. In embodiments, connector 204b may be integrally connected to shaft 202 and/or annular loop 204a using suitable fastening techniques. In some embodiments, connector 204b is monolithically formed with shaft 202. In certain embodiments, connector 204b is separate from shaft 202.

In use, as illustrated by arrow “A1,” a proximal portion 204d of cutting member 204 is manually and/or autonomously drawn proximally and/or extended distally through wire lumen 202a and/or connector 204b to transition annular loop 204a between configurations having a diameter large enough to encircle tissue “T” to at least one additional configuration wherein annular loop 204a cinches tissue “T” therein. Electrosurgical energy “E” may be conducted through annular loop 204a to severe tissue “T” for removal through suction lumen 202b of shaft 202 similar to that described above with respect to electrosurgical instrument 10.

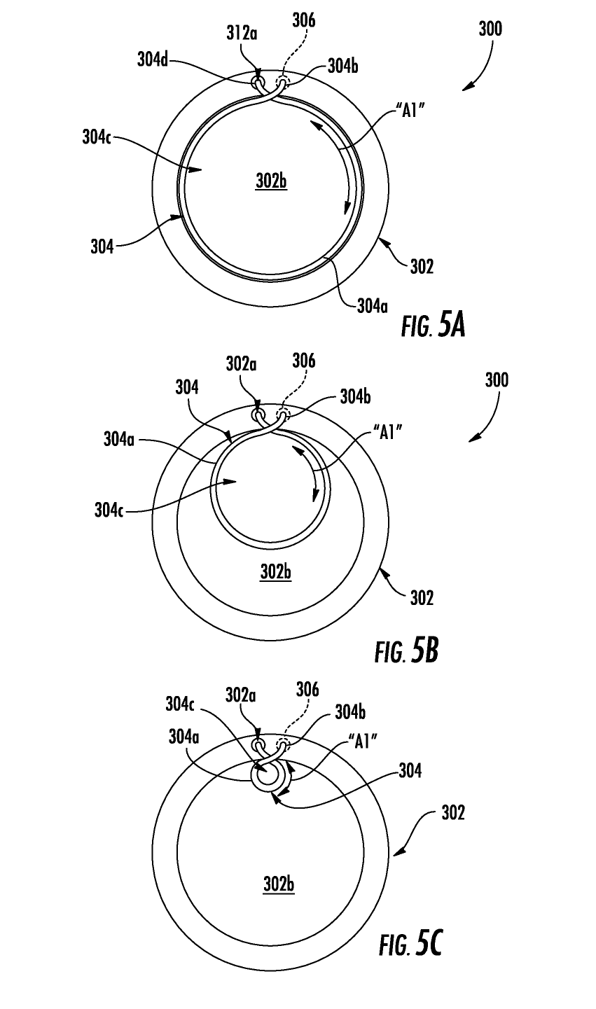

FIGS. 5A-5C illustrate another embodiment an electrosurgical instrument 300 that is similar to electrosurgical instrument 10 and therefore is described herein only to the extent necessary to describe the differences in construction and operation thereof. Electrosurgical instrument 300 includes a shaft 302 and a cutting member 304. Shaft 302 defines a wire lumen 302a that receives a proximal portion 304d of cutting member 304 and suction lumen 302b that is in fluid communication with fluid source 90 (FIG. 1A). Cutting member 304 extends distally to an annular loop 304a and has a fixed end 304b. Annular loop 304a defines a central opening 304c. Fixed end 304b may be coupled to shaft 302 using any suitable fastening technique. In embodiments, fixed end 304b can include a fastener 306 (e.g., a ferrule or the like fastener) to facilitate securement of fixed end 304b to shaft 302. Fastener 306 can be formed of any suitable electrically conductive and/or non-electrically conductive material.

In use, as illustrated by arrow “A1,” proximal portion 304d of cutting member 304 is manually and/or autonomously drawn proximally and/or extended distally through wire lumen 302a to transition annular loop 304a between configurations having a diameter large enough to encircle tissue “T” to at least one additional configuration wherein annular loop 304a cinches tissue “T” therein. Electrosurgical energy “E” may be conducted through annular loop 304a to severe tissue “T” for removal through suction lumen 302b of shaft 302 similar to that described above with respect to electrosurgical instrument 10.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. An electrosurgical instrument for removing tissue, comprising:
a shaft extending to a distal end and defining a lumen configured to communicate with a vacuum source; and
a cutting member supported at the distal end of the shaft, the cutting member adapted to couple to an electrosurgical energy source and configured to form an annular loop, the cutting member selectively transitionable from a first configuration, wherein the annular loop includes a first diameter, to at least one additional configuration, wherein the annular loop includes a second diameter smaller than the first diameter, the annular loop configured to sever tissue within the annular loop.

2. The electrosurgical instrument of claim 1, wherein a portion of the cutting member extends through the shaft and is axially movable relative to the shaft to transition the annular loop from the first diameter to the second diameter.

3. The electrosurgical instrument of claim 1, wherein the annular loop defines an annular cutting surface configured to at least partially circumferentially cut through tissue in response to the transitioning of the annular loop.

4. The electrosurgical instrument of claim 1, wherein the annular loop defines a central opening, the central opening defining a central axis therethrough, the central axis being parallel to, or coaxial with, a longitudinally axis defined by the shaft.

5. The electrosurgical instrument of claim 1, wherein a portion of the cutting member includes an insulative material.

6. The electrosurgical instrument of claim 1, wherein at least a portion of the shaft is formed of an insulative material.

7. The electrosurgical instrument of claim 1, wherein the shaft is at least partially formed from an electrically-conductive material that is insulated from at least the annular loop of the cutting member, the electrically-conductive material of the shaft configured to provide a return path for electrosurgical energy communicated to the annular loop of the cutting member.

8. The electrosurgical instrument of claim 1, wherein the annular loop is configured to electrically communicate with a return pad in electrical communication with the electrosurgical energy source.

9. The electrosurgical instrument of claim 1, wherein the annular loop forms a monopolar electrode.

10. An electrosurgical system for removing tissue, comprising:
an electrosurgical energy source;
a vacuum source;
a shaft extending to a distal end and defining a lumen configured to communicate with the vacuum source; and
a cutting member supported at the distal end of the shaft, the cutting member adapted to couple to the electrosurgical energy source and configured to form an annular loop, the cutting member selectively transitionable from a first configuration wherein the annular loop includes a first diameter, to at least one additional configuration, wherein the annular loop includes a second diameter smaller than the first diameter, the annular loop configured to sever tissue within the annular loop.

11. The electrosurgical instrument of claim 10, wherein a portion of the cutting member extends through the shaft and is axially movable relative to the shaft to transition the annular loop from the first diameter to the second diameter.

12. The electrosurgical instrument of claim 10, wherein the annular loop defines an annular cutting surface configured to at least partially circumferentially cut through tissue in response to the transitioning of the annular loop.

13. The electrosurgical instrument of claim 10, wherein the annular loop defines a central opening, the central opening defining a central axis therethrough, the central axis being parallel to, or coaxial with, a longitudinally axis defined by the shaft.

14. The electrosurgical instrument of claim 10, wherein a portion of the cutting member includes an insulative material.

15. The electrosurgical instrument of claim 10, wherein at least a portion of the shaft is formed of an insulative material.

16. The electrosurgical instrument of claim 10, wherein the shaft is at least partially formed from an electrically-conductive material that is insulated from at least the annular loop of the cutting member, the electrically-conductive material of the shaft configured to provide a return path for electrosurgical energy communicated to the annular loop of the cutting member.

17. The electrosurgical instrument of claim 10, wherein the annular loop is configured to electrically communicate with a return pad in electrical communication with the electrosurgical energy source.

18. The electrosurgical instrument of claim 10, wherein the annular loop forms a monopolar electrode.

19. A method for removing tissue, the method comprising:
encircling tissue with an annular loop disposed at a distal end of a shaft, the annular loop including an initial diameter;
activating the annular loop with electrosurgical energy to circumferentially cut the tissue encircled therein;
transitioning the diameter of the annular loop to facilitate severance of the tissue; and
aspirating the tissue via the shaft.

20. The method of claim 19, further including removing the tissue from a throat with monopolar energy, bipolar energy, or combinations of bipolar and monopolar energy, the tissue including tonsil tissue, adenoid tissue, or combinations of tonsil and adenoid tissue.
